(19) United States
(12) Patent Application Publication
(10) Pub. No.: US 2015/0366460 A1
(43) Pub. Date: Dec. 24, 2015

(54) OBJECT INFORMATION ACQUIRING APPARATUS

(71) Applicant: CANON KABUSHIKI KAISHA, Tokyo (JP)

(72) Inventors: Minoru Ohkoba, Sagamihara-shi (JP); Shigeru Ichihara, Tokyo (JP)

(21) Appl. No.: 14/737,753

(22) Filed: Jun. 12, 2015

(30) Foreign Application Priority Data
Jun. 20, 2014 (JP) 2014-127527

(51) Int. Cl. A61B 5/00

(52) U.S. Cl. CPC A61B 5/0095; A61B 5/7203; A61B 2576/00

(57) ABSTRACT

Provided is an object information acquiring apparatus, comprising: a laser light source configured to irradiate laser light onto an object; a detector configured to detect a part of the laser light from the laser light source; a receiver configured to receive an acoustic wave that propagate from the object, based on the irradiation of the laser light; and an acquisition unit configured to acquire information relating to the object, based on a reception result of the receiver and a detection result of the detector, wherein the detector detects anomalous emission from the laser light source.

### BACKGROUND OF THE INVENTION

1. Field of the Invention

The present invention relates to an object information acquiring apparatus.

2. Description of the Related Art

Development of a photoacoustic tomography apparatus (object information acquiring apparatus) for medical purposes using a short pulse oscillation laser is progressing (Manohar et al, “Region-of-interest breast studies using the Twente Photoacoustic Mammoscope (PAM)” Proc. Of SPIE, Vol. 6437, 643702). Photoacoustic tomography (PAT) is a technique for forming images by irradiating a pulse laser (at several tens to several hundred nano-seconds) onto a measurement segment, receiving a photoacoustic wave generated in the segment using a probe, and processing the acquired receive signals. The PAT allows analysis of biological functions by the spectral measurement based on the absorption coefficient of the biological tissue.

To generate short pulse light used for measuring an acoustic wave, a laser using a Q switch is used. Q switch oscillation is a technique to oscillate a high output and short pulse laser light by controlling a Q value of the resonator performance index, which is a half-width function of an oscillation pulse. The laser oscillation in this case is called giant pulse oscillation.

An apparatus that acquires object information by irradiating laser light based on the laser oscillation using such a Q switch has been proposed (Japanese Patent Application Laid-Open No. 2013-89680).

### SUMMARY OF THE INVENTION

However in the case of a laser apparatus using a Q switch, anomalous emission such as pre-lasing occurs when characteristics of the apparatus are unstable. One challenge is to detect and decrease the anomalous emission such as pre-lasing. Because of the anomalous emission, an acoustic wave signal is generated from the biological tissue. This acoustic wave signal, due to the anomalous emission, becomes noise when an acoustic signal is analyzed, interrupting the acquisition of accurate biological information (object information). Further, the pulse width of one giant pulse disperses, and a desired acoustic wave signal cannot be acquired. Moreover, generation of anomalous emission such as pre-lasing is strongly correlated with ambient temperature of the laser apparatus. However if generation of anomalous emission such as pre-lasing is suppressed by controlling the temperature of the laser apparatus, the object information acquiring apparatus becomes large, and manufacturing cost increases.

With the foregoing in view, it is an object of the present invention to provide an object information acquiring apparatus where influence of anomalous emission is minimized.

To achieve the object, the present invention uses the following configuration. In other words, an object information acquiring apparatus, comprising: a laser light source configured to irradiate laser light onto an object; a detector configured to detect a part of the laser light from the laser light source; a receiver configured to receive an acoustic wave that propagate from the object, based on the irradiation of the laser light; and an acquisition unit configured to acquire information relating to the object, based on a reception result of the receiver and a detection result of the detector, wherein the detector detects anomalous emission from the laser light source.

The present invention also uses a following configuration. In other words, an object information acquiring apparatus, comprising: a laser light source configured to irradiate laser light onto an object; a detector configured to detect a part of the laser light from the laser light source; a determination unit configured to determine whether anomalous emission is included in the laser light, based on the detection result of the detector; a receiver configured to receive an acoustic wave that propagate from the object, based on the irradiation of the laser light; and an acquisition unit configured to acquire information relating to the object, based on a reception result of the receiver and a determination result of the determination unit.

The present invention also uses a following configuration. In other words, an object information acquiring apparatus, comprising: a laser light source configured to irradiate laser light onto an object; a detector configured to detect a part of the laser light from the laser light source; a receiver configured to receive an acoustic wave that propagate from the object, based on the irradiation of the laser light; and an acquisition unit configured to acquire information relating to the object, based on a reception result of the receiver and a detection result of the detector, wherein the acquisition unit acquires information on the object using the reception result of the receiver, excluding a reception result, which is acquired when the detection result of the detector indicates anomalous emission.

As mentioned above, the present invention can provide an object information acquiring apparatus where influence of anomalous emission is minimized.

Further features of the present invention will become apparent from the following description of exemplary embodiments with reference to the attached drawings.

### BRIEF DESCRIPTION OF THE DRAWINGS

FIG. 1A is a diagram depicting Example 1 of an object information acquiring apparatus of the present invention;

FIG. 1B is a diagram depicting a laser light source according to Example 1 of the present invention;

FIG. 2A to FIG. 2D are diagrams depicting a relationship of normal oscillation, giant pulse oscillation and pre-lasing;

FIG. 3 is a diagram depicting a positional relationship of a laser light sensor with respect to elements according to Example 1;

FIG. 4A and FIG. 4B are graphs showing a typical irradiation amount acquisition result according to Example 1;

FIG. 5 is a diagram depicting a laser light sensor of an object information acquiring apparatus according to Example 2 of the present invention;

FIG. 6 is a diagram depicting a laser light sensor of an object information acquiring apparatus according to Example 3 of the present invention;

FIG. 7 is a diagram depicting a laser light sensor unit according to Example 4 of the present invention;

FIG. 8 is a diagram depicting a laser light sensor unit according to Example 5 of the present invention;

FIG. 9 is a diagram depicting a laser light sensor unit according to Example 6 of the present invention; and

FIG. 10 is a diagram depicting a comparative technique with respect to the present invention.

### DESCRIPTION OF THE EMBODIMENTS

Embodiments of the present invention will now be described with reference to the drawings. As a rule, a same composing element is denoted with a same reference number, for which redundant description is omitted. Calculation expressions, calculation procedures or the like to be described in detail herein below should be appropriately changed depending on the configuration of the apparatus to which the present invention is applied and various conditions, and are not intended to limit the scope of the invention to the following description.

An object information acquiring apparatus of the present invention includes an apparatus utilizing a photoacoustic effect, which receives an acoustic wave generated in an object by irradiating light (electromagnetic wave) such as near infrared, onto the object, and acquires the object information as image data. In the case of the apparatus utilizing the photoacoustic effect, the object information to be acquired refers to the generation source distribution of the acoustic wave generated by light irradiation, the initial sound pressure distribution in the object, the absorption density distribution or absorption coefficient distribution of light energy derived from the initial sound pressure distribution, or the substance concentration distribution constituting the tissue. Examples of the substance concentration distribution are: an oxygen saturation distribution; a total hemoglobin concentration distribution; and an oxy/deoxy hemoglobin concentration distribution.

Characteristic information, which is the object information at a plurality of locations, may be acquired as two-dimensional or three-dimensional characteristic distribution. The characteristic distribution can be generated as image data that indicates the characteristic information inside the object. The acoustic wave referred to in the present invention is typically an ultrasound wave, that includes a sound wave and a light-induced ultrasound wave. An acoustic wave generated by the photoacoustic effect is called a photoacoustic wave or a light-induced ultrasound wave. An acoustic detector (e.g. probe) receives an acoustic wave generated or reflected inside the object.

In actual anomalous emission, the total output is lower than a giant pulse emission. Therefore a sensor, of which sensitivity and temporal resolution are very high, must be used to detect only an anomalous emission by resolving the emission with respect to time. This means that the sensor becomes expensive. Further, in the case of pre-lasing, which is an example of anomalous emission, the period from the start of generation of the oscillation to sending a Q switch OFF signal for starting the generation of a giant pulse is short, several nanoseconds to several tens of nanoseconds. In practical terms, it is very difficult to negate generating the oscillation of a giant pulse during this short period. Techniques to solve these problems will now be described herein below.

### Example 1

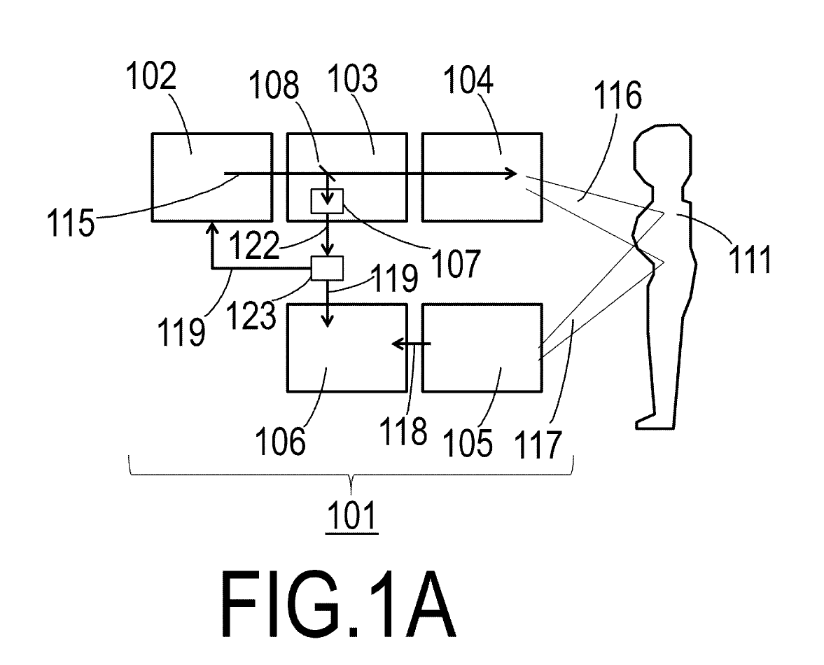

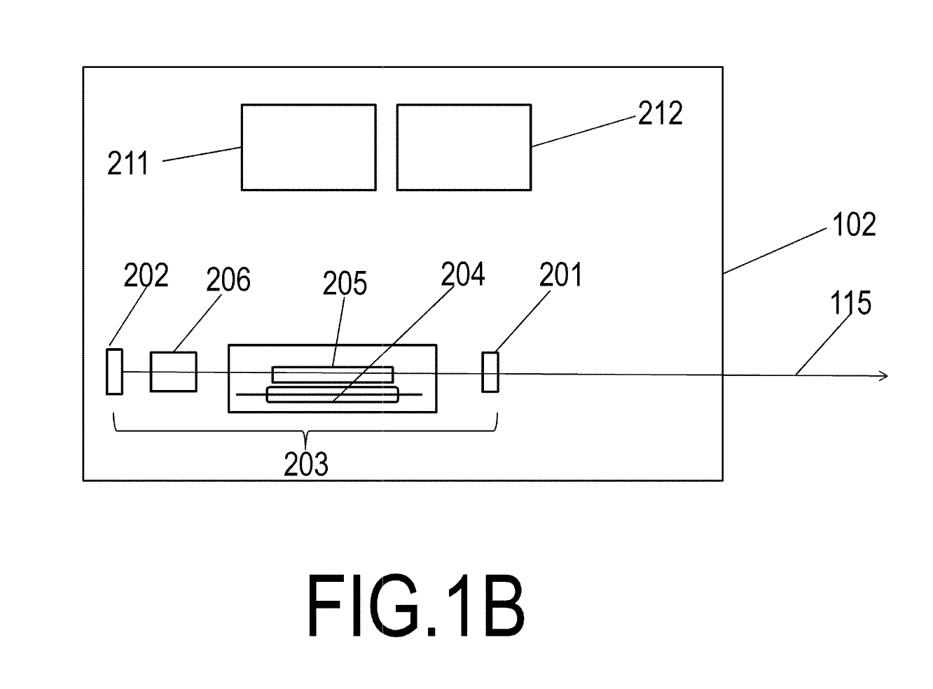

FIG. 1A is a diagram depicting Example 1 of the object information acquiring apparatus of the present invention. The object information acquiring apparatus 101 includes a laser light source 102. A light transmission optical system 103, a light irradiation optical system 104 which is an irradiation unit, an acoustic wave receiver 105, and an acoustic wave signal processing unit (an acoustic wave signal processor) 106 are also included. Furthermore, a laser light sensor 107 which is a detector, a branch mirror 108, an object 111 and an irradiation light 116 are also illustrated in FIG. 1A. Furthermore, an intensity detection signal 122, an acoustic wave signal 117, an electric signal 118, a determination unit 123, and an anomalous emission determination signal, that is a determination result 119, outputted from the determination unit 123, are also illustrated in FIG. 1A.

The object information acquiring apparatus 101 is an apparatus to acquire information inside the object 111 using a photoacoustic wave signal. A part of the energy of the light propagated inside the object 111 is absorbed by an absorber (sound source), such as hemoglobin in blood. Then the acoustic wave signal 117 is generated by the thermal expansion of the light absorber, and the acoustic wave signal 117 propagates inside the object. The propagating acoustic wave signal 117 is converted into the electric signal 118 by a probe disposed in the acoustic wave receiver 105, and is transferred to the acoustic wave signal processing unit 106, which is an acquisition unit (an acquisition device). The electric signal 118 is converted into optical characteristic value distribution information or the like inside the object 111 by the acoustic wave signal processing unit 106, and becomes object information. The generated object information includes the optical characteristic value distribution and absorption coefficient distribution, as well as initial sound pressure distribution, substance concentration and oxygen saturation based thereon. Image data for displaying an image reconstructed based on this information can also be included.

The laser light source 102 provides light that is transmitted through a biological body, which is the object 111, so as to appropriately transmit a photoacoustic signal generated by a measurement target, such as hemoglobin in a blood vessel. To increase the signal accuracy of the photoacoustic signal, that is the acoustic wave signal 117, light having high power must propagate to the object 111. The laser light is used for this reason. Furthermore, the light must reach the measurement target hemoglobin or the like without being absorbed in the object 111 very much, hence the wavelength of the light that can easily propagate in the object 111 is limited, and light having a 500 nm to 1200 nm wavelength characteristic in particular is used. For this, an alexandrite laser or a titanium sapphire laser is preferably used. Furthermore, in order to improve the signal accuracy of the acoustic wave signal 117, a pulse light with short pulse width, several tens to several hundreds of nanometers, is used for the laser light 115. To generate this laser light having high power and short pulse width, a laser that oscillates with giant pulses using the Q switch is preferably used. The laser light source 102 may be integrated into the object information acquiring apparatus 101, or may be installed externally.

The light transmission optical system 103 has a function to propagate light from the laser light source 102 to the light irradiation optical system 104. Distance is generated between the laser light source 102 and the light irradiation optical system 104 depending on the configuration. Because of this distance, the laser light 115 spreads. Therefore in order to suppress this spreading, lenses or the like are disposed on the optical path of the laser light 115. If the lenses or the like cannot be disposed on a straight line because of the positional relationship of the laser light source 102 and the light irradiation optical system 104, the traveling direction of the laser light 115 is adjusted by disposing a reflection mirror or the like. Thereby the laser light is guided to a desired location. If necessary, the laser light is guided to a timing trigger which measures a light transmission timing that is required by the acoustic wave signal processing unit 106, or to such a measurement apparatus as a laser light sensor 107 of the present invention. For this purpose, the branch mirror 108 is disposed on the optical path and the branched light is guided to these measurement apparatuses. Optical fibers may be used for some part of the light transmission in the light transmission optical system 103.

The light irradiation optical system 104 forms the irradiation light 116 from the laser light 115 propagated by the light transmission optical system 103, and irradiates the light 116 onto a measurement target segment in the object 111. For this, the light irradiation optical system 104 plays a role of transforming the light quantity distribution of the laser light 115 to an appropriate light quantity distribution for the object 111, such as spreading the laser light 115. The light irradiation optical system 104 includes a lens and a diffusion plate in order to form the irradiation light 116 by expanding or diffusing the laser light 115 appropriately, so that the acoustic wave signal 117 is ideally acquired and the irradiation amount to the object 111 (biological body) does not exceed a specified value.

The acoustic wave receiver 105 has a probe that receives the acoustic wave signal 117. The probe receives the acoustic wave signal 117 which is generated on the surface, inside of the biological body or the like by the pulsed light (irradiation light 116), and converts the acoustic wave into an analog electric signal 118. The probe can be any probe that can receive an acoustic wave signal, such as a probe using piezoelectric phenomena, a probe using the resonance of light, or a probe using the change in electrostatic capacitance. The probe of this embodiment is typically a probe where a plurality of reception elements (e.g. piezoelectric elements) are one-dimensionally or two-dimensionally disposed, or are spirally disposed on the base of a bowl-shaped fixed component. If such multi-dimensionally arrayed elements are used, the acoustic wave signal 117 can be simultaneously received at a plurality of locations. As a result, the measurement time can be shortened. And to increase a number of measurement locations by a probe, the probe may be scanned so as to receive the acoustic wave signal 117 at a plurality of locations. The acoustic wave signal 117 received by the probe is converted into the electric signal 118, and is then used for generating the characteristic information by the acoustic wave signal processing unit 106.

The acoustic wave signal processing unit 106 is constituted by an information processor, such as a computer, and circuits, and processes and computes the electric signal 118. The acoustic wave signal processing unit 106 includes a conversion unit, such as an A/D convertor, which coverts an electric signal acquired by the probe (analog signal) into a digital signal. It is preferable that the conversion unit can simultaneously process a plurality of signals. Thereby the time required to generate an image (image reconstruction) can be decreased. The converted digital signal is stored in memory in the acoustic wave signal processing unit 106. The acoustic wave signal processing unit 106 generates object information, such as optical characteristic value distribution, using the data or the like stored in this memory, by back projection in a time domain, for example.

FIG. 1B is a diagram depicting the laser light source 102 of Example 1 of the present invention. As illustrated in FIG. 1B, the laser light source 102 of the present invention includes: a laser resonator 203 constituted by two reflectors (an output mirror 201 and a reflection mirror 202); a laser controller 211 which is a control unit; and a laser power supply 212 that supplies power to the light source 102. Wires of the laser controller 211 and the laser power supply 212 or the like are omitted. Here the laser controller 211 is disposed in the light source 102. In other words, the laser controller 211 is disposed in the previous stage of the detector 107, which is disposed in the light transmission optical system 103. The laser controller 211 may include information processors and circuits of a CPU, MPU, memory or the like.

An excitation unit (an excitation device) 204, a laser medium 205 and a Q switch 206 are disposed inside the resonator. The laser controller 211 controls the voltage that is applied to the excitation unit 204 and the Q switch 206. The excitation unit 204 uses a flash lamp or a semiconductor laser, and if a rod type laser medium 205 is used, the excitation unit 204 optically excites the laser medium 205 from the side surface. For the Q switch 206, Pockels cells, which are optical crystals of potassium dihydrogen phosphate (KDP), potassium deuterium phosphate (DKDP) or the like, are used. Pockels cells are elements where the refractive index changes with anisotropy in proportion to the electric field strength, and the polarizing direction of the transmitted light rotates. Therefore Pockels cells are widely used to acquire giant pulsed light, of which oscillation pulse width is small and output intensity is high. Although the pulse width is different depending on the type of laser medium, resonator length and optical resonance state, a 100 ns or less pulse width is acquired. If Nd:YAG crystals or alexandrite crystals are used for the laser medium, the configuration in FIG. 1B is used. In the case of a titanium sapphire laser, a second harmonic of the Nd:YAG laser is used for the excitation source of the titanium sapphire crystals. In the titanium sapphire laser, the present invention is applied to the Nd:YAG laser portion, which is the excitation source. In this description, an alexandrite laser, where the laser medium is excited by a flash lamp, will be described as a reference. The alexandrite laser has grains in a 700 nm to 800 nm range, and becomes a wavelength variable laser by disposing a wavelength selection mechanism constituted by a birefringent filter between the laser medium 205 and Pockels cells (the Q switch 206) inside the resonator.

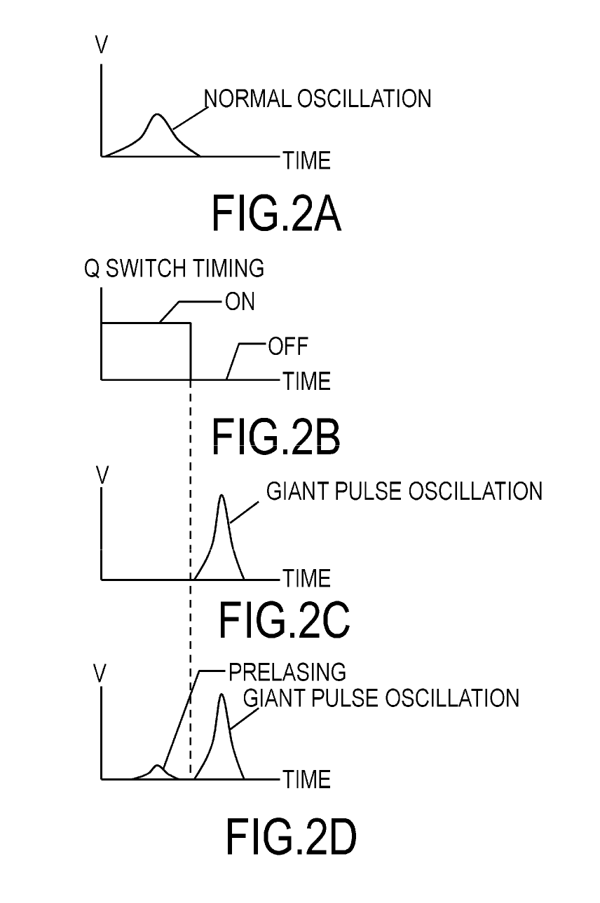

FIG. 2A to FIG. 2D are conceptual diagrams depicting the relationship of the normal oscillation, giant pulse oscillation and anomalous emission such as pre-lasing. Here pre-lasing will be described with reference to FIG. 2. Anomalous emission such as pre-lasing is a phenomena that is generated when oscillation using the Q switch is performed. Prior to describing pro-lasing, normal oscillation that does not use the Q switch, and the giant pulse oscillation that uses the Q switch will be described first.

FIG. 2A shows a temporal transition of normal oscillation. In the normal oscillation, a predetermined inverted distribution energy is stored in crystals by the excitation light, and laser light is oscillated from the resonator when the stored energy reaches a threshold energy. In the case of the normal oscillation, the pulse width of oscillation is wider than the later mentioned giant pulse oscillation.

FIG. 2B shows a temporal transition of the ON/OFF driving of the Q switch, and FIG. 2C shows a temporal transition of the oscillation using the Q switch based on this ON/OFF driving. In the case of a laser which performs oscillation using a Q switch, the Q switch is disposed inside the resonator, and oscillation is suppressed for a predetermined period (several tens μs to several hundreds μs) by the Q switch. During this period, the inverted distribution energy is stored in the crystals by the excitation light, whereby energy higher than the threshold energy is forcibly stored. A laser light of which output is high and pulse width is short is oscillated by the Q switch, clearing suppression of resonance (increasing Q value of resonator) after the predetermined period elapsed. This is called giant pulse oscillation.

FIG. 2D shows a temporal transition of general oscillation in the case when anomalous emission such as pre-lasing is generated. Pre-lasing oscillation refers to a phenomena where a part of the energy stored before the giant pulse oscillation leaks in a laser which oscillates using the Q switch. There are a variety of different causes for this, such as a mechanism of members constituting the Q switch, and the optical characteristics of other composing members in the resonator. The Q switch laser was originally designed targeting a mechanism that suppresses pre-lasing and accurately generates a giant pulse, hence if pre-lasing oscillation occurs, deviating from this target, then oscillation energy and pulse width often become unstable. In the oscillation of one pulse, the pre-lasing is generated when the Q switch is in the ON state. Then the Q switch turns OFF and the giant pulse oscillation is generated. Therefore a laser light to be generated has a pulse width that is different from the case when the giant pulse is accurately oscillated.

Particularly in the case of a solid-state laser that uses a rod type laser medium, if anomalous emission/oscillation such as pre-lasing is generated in a center area of the rod where excitation efficiency is high, a giant pulse oscillation is continuously generated triggered by this oscillation, concentrated at the center. As a result, strong oscillation having a special intensity distribution, which is described later, is generated. As a Q switch, a device that induces refractive index anisotropy of Pockels cells or the like in the electric field is used to change the polarizing direction of the reciprocating light. The resonance is suppressed using this characteristic of the Q switch. If an optical shutter to suppress such resonance is used, the polarizing state of the giant pulse light and the polarizing state of the anomalous emission such as pre-lasing differ. The characteristic of the Q switch is utilized in an example described later.

The laser light sensor 107 disclosed in the present invention does not detect only feeble emission when the Q switch is ON (resonance suppression period). In other words, the laser light sensor 107 acquires oscillation intensity such that both the feeble emission, which is an anomalous emission such as pre-lasing, and the giant pulse oscillation, which is emitted triggered by feeble transmission (pre-lasing) after the Q switch is turned OFF, are included on the time axis. The laser light sensor 107 outputs a signal in accordance with the acquired intensity in the detection result to the determination unit 123 in FIG. 1A. The determination unit 123 is disposed to identify the emission generated due to the anomalous emission such as pre-lasing, out of the outputted signals. Thereby generation of the anomalous emission such as pre-lasing can be detected well, even if a laser light sensor 107, of which time resolution is not very high, is used. Here a “laser light sensor of which time resolution is not very high” refers to, for example, a sensor that can detect the intensity of light only in a period from when anomalous emission such as pre-lasing is generated to when the giant pulse oscillation ends in FIG. 2D. A “laser light sensor of which time resolution is high” on the other hand, refers to a sensor that can detect the intensity of a laser light in a period from the beginning of anomalous emission such as pre-lasing to the end thereof in FIG. 2D.

A method for controlling the object information acquiring apparatus or the laser apparatus included therein in the case when anomalous emission such as pre-lasing is generated will be described. If a cause of generating anomalous emission such as pre-lasing is presumed, a control method for minimizing this cause is introduced, or a method of stopping the object information acquiring apparatus or the laser apparatus included therein is used. A case when the cause is presumed and retraceable, such as a case when a member constituting the Q switch is a member of which refractive index anisotropy is changed by applying voltage, as in the case of Pockels cells, is considered. In this case, the optimum voltage to be applied to Pockels cells may be shifted by the influence of temperature or the like of the laser apparatus. As a result, anomalous emission such as pre-lasing may be generated. In such a case, generation of anomalous emission such as pre-lasing can be suppressed by changing the voltage to be applied to the Pockels cells as a control of the laser apparatus. By disposing such a control mechanism, a stable object information acquiring apparatus can be provided. If the anomalous emission such as pre-lasing is accidently generated due to the unstable operation of the Q switch or the like, information, to identify whether the emission is an emission that includes anomalous emission such as pre-lasing or an emission that does not include anomalous emission such as pre-lasing, is also included in the output data. Then if only the acoustic wave signals acquired by the emission which does not include anomalous emission such as pre-lasing is used for image reconstruction, then noise of the reconstructed image can be removed. It is also possible to output, along with the reconstructed image, information that the image was constructed based on the laser light where anomalous emission such as pre-lasing was generated.

Now FIG. 1A is once again referred to. To generate a pulsed light of which pulse width at a 750 nm wavelength is 100 nsec and the repetition frequency is 20 Hz as the laser light source 102, an aperture as a mode selector is disposed inside the resonator as a mode selector. A lamp excitation type or a Q switch oscillation type alexandrite light laser light source, which generates a multi-mode pulsed light of which beam profile is φ5 mm, is used. For the output, one pulse is emitted at 300 mJ. As the light transmission optical system 103, a convex lens (f=1000 mm), for propagating the laser light 115 as approximately parallel light, is disposed on the optical path. As the acoustic wave receiver 105, a probe is disposed on an array. The object 111 is a biological body, such as a breast. The branch mirror 108 is disposed in a subsequent stage of the light source 102 so that the reflectance becomes 1% at a 45° reflection, and branches the laser light 115. 1% of the laser light after branching is guided to the laser light sensor 107 according to the present invention. The laser system of this example includes an air conditioning system to stabilize the apparatus.

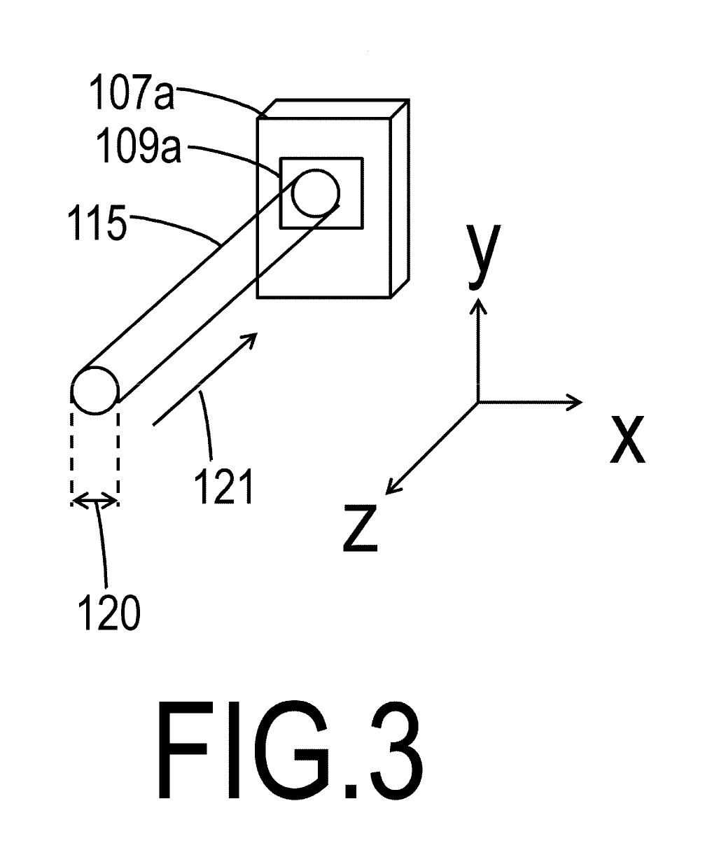

FIG. 3 is a diagram depicting a positional relationship of the laser light sensor with respect to elements according to Example 1. The laser light sensor 107a used for this example will be described with reference to FIG. 3. FIG. 3 shows the laser light sensor 107a, a photo acceptance unit 109a, a laser light 115, a width 120 of the distribution of the laser light and the traveling direction 121 of the laser. For the laser light sensor 107a of this example, a beam profiler having a 10 mm×10 mm sized photo acceptance unit 109a is used. There are 100 elements existing in a 10 mm distance that are arranged such that the laser traveling direction 121 is the z axis direction, and the photo acceptance unit 109a is on the xy plane, that is, a plane perpendicular to the z axis direction. In other words, the photo acceptance unit 109a is an area sensor that can measure the intensity distribution of the laser light on the xy plane, for example. Further, these elements are arranged so that the center of the intensity distribution of the laser light 115 comes to the center of the photo acceptance unit 109a. By this arrangement, each of the 100×100 elements acquires the irradiation energy (intensity) of each pulse, including anomalous emission such as pre-lasing and giant pulse. Then the laser light sensor 107a transmits the acquired result to the determination unit 123 in FIG. 1A. This transmission method may be radio communication or may be by disposing wires and transmitting the acquired result as voltage or current signals.

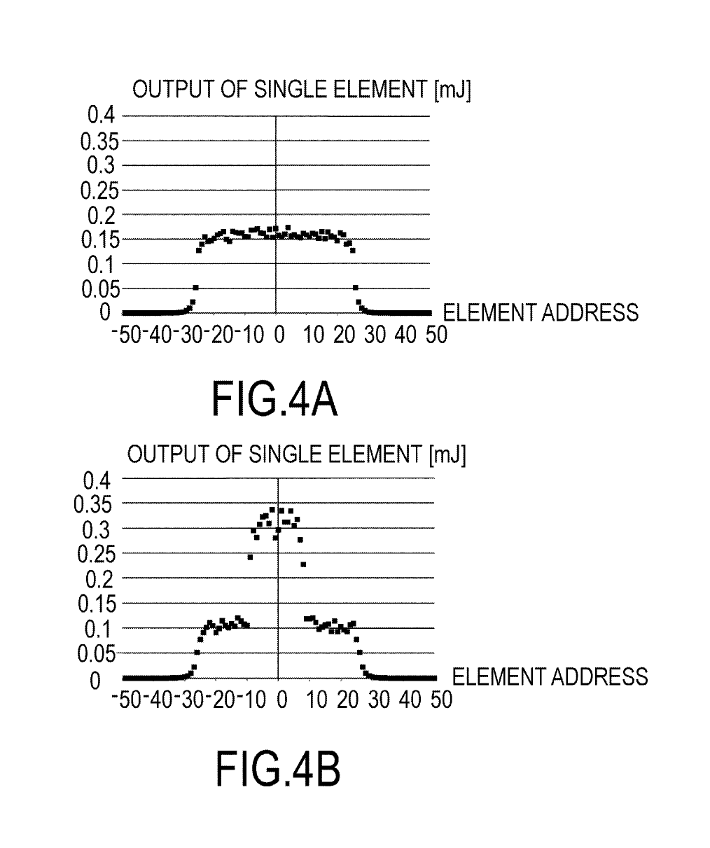

FIG. 4A to FIG. 4B are graphs showing the typical irradiation amount acquisition result according to Example 1. In FIG. 4A and FIG. 4B, the abscissa indicates the address (coordinate) in the x direction in FIG. 3, and the ordinate indicates the energy of the laser light 115 entering each element. The position at coordinate x=0 matches with the center of the width 120 of the distribution of the laser light in FIG. 3. Both FIG. 4A and FIG. 4B were acquired when the address in the y direction is at the center, and show a cross-sectional profile sectioned at the center of the beam profile. FIG. 4A is a graph showing a typical giant pulse emission energy. FIG. 4B is a graph showing emission energy when the emission energy of both anomalous emissions such as pre-lasing and giant pulses is acquired for a predetermined time. The predetermined time refers to the time that includes both time when anomalous emission such as pre-lasing is generated and time when a giant pulse is generated in one pulse. A number of elements in a φ5 mm range, where a giant pulse is generated (a range where the element addresses are −50 to 50), corresponds to approximately 2000, and about 0.15 mJ of energy is measured for each element by the laser light sensor 107a.

The range of the emission energy (range where intensity of laser light 115 is distributed) when the emission energy including both the anomalous emission such as pre-lasing and the giant pulse is acquired, disperses. However this energy is observed concentrating in a range that is about φ2 mm (range of which element addresses are −20 to 20), which is narrower than the range of φ5 mm where the giant pulse is generated (a part of the range where the intensity of the laser light 115 is distributed). When the anomalous emission such as pre-lasing is generated, the total of the emission energy observed by each element remains 300 mJ. In other words, this is the same as the total emission energy of the typical giant pulses observed by each element in FIG. 4A. However, in the range where the anomalous emission such as pre-lasing concentrates, that is about φ2 mm, the laser light sensor 107a observes 0.3 mJ output per element. In other words, in this range of about φ2 mm, the emission energy observed by each element, when anomalous emission such as pre-lasing is generated as shown in FIG. 4B, is higher than the emission energy observed by each element shown in FIG. 4A. Further, as shown in FIG. 4B, a peak of the output of one element exists near the element address 0.

A criteria is set in the determination unit 123 to determine whether anomalous emission such as pre-lasing is generated or not in a laser having such an emission energy distribution characteristic. In other words, a criteria is set so as to determine that anomalous emission such as pre-lasing is generated if an average value, calculated by dividing the total energy of each element in the φ2 mm range by a total number of elements in the φ2 mm range, is 0.25 mJ or more. Thereby anomalous emission such as pre-lasing is accurately detected. In other words, the determination unit 123 compares the determination threshold, which is a predetermined value, with the average value based on the detected result by the laser light sensor 107a. Then, if the average value exceeds the determination threshold in the comparison result, the determination unit 123 determines that anomalous emission such as pre-lasing is generated, and outputs this determination result 119. If the average value does not exceed the determination threshold in the comparison result, on the other hand, the determination unit 123 determines that anomalous emission such as pre-lasing is not generated, and outputs this determination result 119. Possible output destinations are the acoustic wave signal processing unit 106 and the laser light source 102.

The determination unit 123 is a single block in FIG. 1A, but may be disposed inside the light transmission optical system 103. The determination unit 123 may be disposed as a unit that is separate from the laser light sensor 107, or may be integrated with the laser light sensor 107. The laser light sensor 107 may have the function of the determination unit 123, and in this case, the laser light sensor 107 detects the spatial distribution of the intensity of the laser light. Based on this detection, the determination unit 123 may also detect the anomalous emission, and send this detection result (detection content) to the acoustic wave signal processing unit 106 and the laser light source 102 as the determination result 119.

In a laser having this emission energy distribution characteristic, it was determined whether anomalous emission such as pre-lasing is generated or not by monitoring the energy in the φ2 mm range, and anomalous emission such as pre-lasing was detected accurately. Moreover, one of the causes of the generation of anomalous emission such as pre-lasing is the rise in temperature of the laser system. Therefore, the control function to suppress the generation of anomalous emission such as pre-lasing by the laser controller dropping the air conditioning temperature of the laser system by 0.1° C. was included. Thereby an object information acquiring apparatus that suppresses the destabilization of the giant pulses was created. Further, by using the above-mentioned sensor configuration, the generation of anomalous emission such as pre-lasing was easily detected, even when the time resolution of the laser light sensor 107 is not very high.

### Example 2

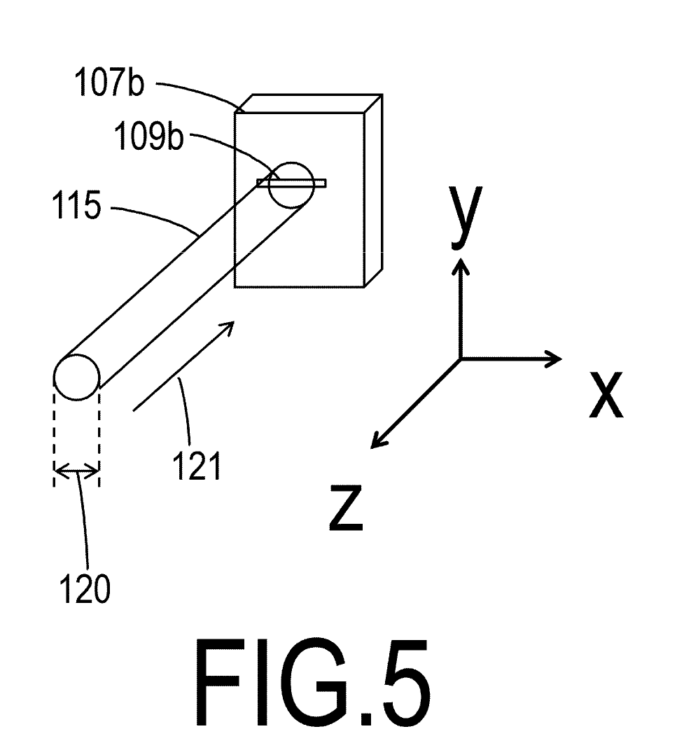

FIG. 5 is a diagram depicting a laser light sensor of an object information acquiring apparatus according to Example 2 of the present invention. A composing element the same as Example 1 is denoted with a same number, for which description is omitted unless necessary. The laser light sensor of Example 1 is an area sensor, that senses the laser light intensity distribution two-dimensionally on the xy plane. However, a one-dimensional line sensor 109b as shown in FIG. 5 may be used for a photo acceptance unit 109b. This photo acceptance unit 109b is divided into 100 elements. Since the φ2 mm range corresponds to about 20 elements of Example 1, the measured value is about 20 times that of Example 1. This means that the energy when a giant pulse is generated is 3.0 mJ, and the energy when an anomalous emission such as pre-lasing is generated is 6.0 mJ. This is equivalent to the result in FIG. 4 of Example 1. In other words, the intensity of the laser light increases on the line from the edge side of the laser width 120 to the center side of the laser width 120. Even if this line sensor 109b is used, the laser light intensity distribution, including the anomalous emission such as pre-lasing and the giant pulse, can be acquired. Particularly when the line sensor 109b is disposed in a certain way, the profile of the intensity distribution acquired by the line sensor 109b becomes close to that in FIG. 4B. This is the case when the line sensor 109b is disposed such that the intensity of the laser light 115 on the plane perpendicular to the traveling direction 121 of the laser light 115 comes to the center of the distribution range.

Since a configuration other than the sensor 107b can be the same as Example 1, an object information acquiring apparatus, where the influence of anomalous emission such as pre-lasing is minimized, can be provided. Further, cost reduction can be expected because accuracy is similar to the laser light sensor 107a of Example 1, and a number of photo acceptance units is less than that of the laser light sensor 107a.

The configuration is not limited to this, and signals based on the intensity of the laser light 115 acquired by the elements in the φ2 mm range, out of the photo acceptance unit 109a of the laser light sensor 107a of Example 1, may be integrated by wiring or the like. And the signals acquired by the photo acceptance unit 109a outside φ2 mm range are not used. Thereby an effect similar to the laser light sensor 107b of Example 2 can be demonstrated.

### Example 3

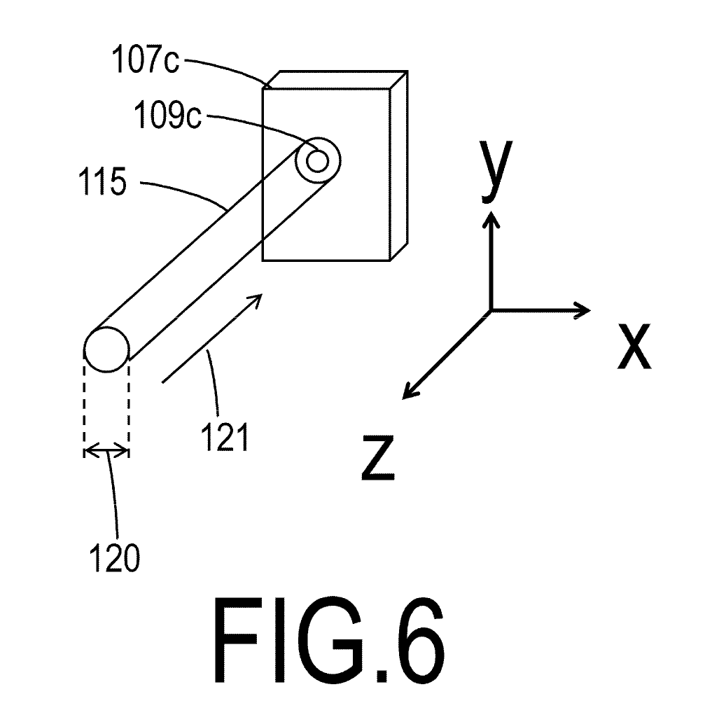

FIG. 6 is a diagram depicting a laser light sensor of an object information acquiring apparatus according to Example 3 of the present invention. A composing element the same as Example 1 is denoted with a same number, for which description is omitted unless necessary. The laser light sensor 107c detects only laser light in a range smaller than the laser light intensity distribution width shown in FIG. 4. In other words, a photo acceptance unit 109c is formed to have a size that approximately matches with the φ2 mm range, so as to detect the laser light intensity in a range where the element addresses in FIG. 4 are in the φ2 mm range. The shape of the photo acceptance unit 109c of this example is different from that of the photo acceptance unit 109a of Example 1. The photo acceptance unit 109c of this example is located at the center of the intensity distribution of the laser light 115, and the photo acceptance unit 109c is an undivided single light intensity sensor that detects the intensity of the laser light. This sensor may be a single plane-shaped area sensor or a single line-shaped line sensor. The φ2 mm range corresponds to about 310 elements of Example 1. This means that the measured value becomes about 310 times that of Example 1. In other words, the energy when a giant pulse is generated is 47 mJ, and the energy when anomalous emission such as pre-lasing is generated in 93 mJ. As feed forward control, a mechanism is disposed so as to attach a value, calculated by dividing the intensity of the laser light 115 detected by the sensor 107c by 310 (an approximate value corresponding to the number of elements) to the photo acoustic signal data, and output this value with the photo acoustic signal data. As mentioned above, the intensity of laser oscillation of one pulse that is generated when anomalous emission such as pre-lasing is generated concentrates at the center portion of the width 120 of the intensity distribution of the laser light 115. To detect the concentrated portion accurately, a power meter, for example, which has the photo acceptance unit 109c that measures only the center portion, is preferably used as the laser light sensor 107c.

Using this configuration, anomalous emission such as pre-lasing is accurately detected and the image is reconstructed after omitting data acquired based on the laser light including anomalous emission such as pre-lasing. As a result, an object information acquiring apparatus that can acquire accurate data can be created.

### Example 4

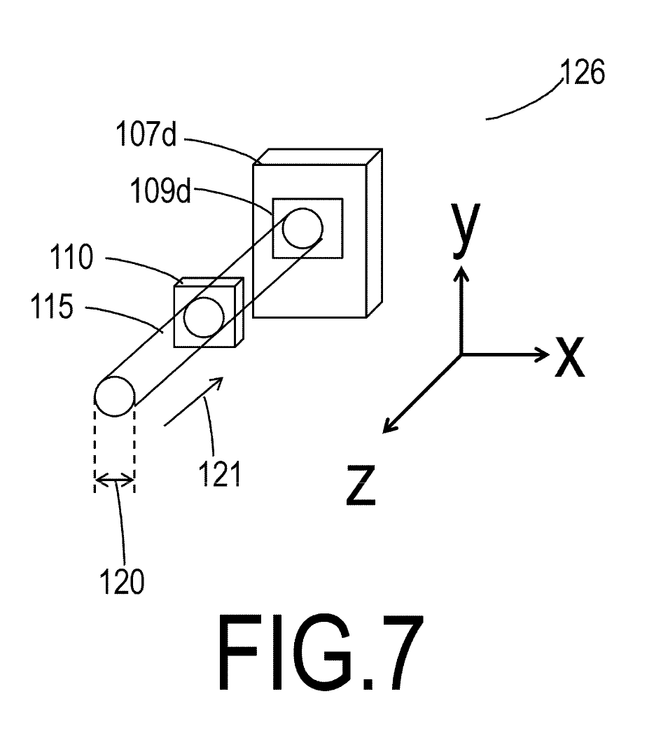

FIG. 7 is a diagram depicting a laser light sensor unit according to Example 4 of the present invention. A composing element the same as Example 1 is denoted with a same number, for which description is omitted unless necessary. A laser light sensor unit 126 of this example has the laser light sensor 107d of Example 2 and a polarizing plate 110 which is a polarizing unit disposed on the front surface of the laser light sensor 107d for detecting the polarized light. An element unit 109d is an undivided single element sensor. The polarizing plate 110 is disposed in a direction where S-polarized light is intensively transmitted. Here the giant pulse is a P-polarized emission. The anomalous emission such as pre-lasing generated by this configuration, on the other hand, is oscillated as an S-polarized light because this light is allowed to oscillate when the Q switch is ON. This sensor 107d receives the anomalous emission such as pre-lasing transmitted through the polarizing plate 110. Then as feed forward control, a mechanism is disposed so as to attach an energy value of anomalous emission such as pre-lasing that is outputted as the reception result to the photo acoustic signal data for each pulse, and output the energy value with the photoacoustic signal data. In other words, the Q switch which includes an element utilizing electric refractive index anisotropy, such as Pockels cells, has a characteristic to polarize anomalous emission such as pre-lasing, and this example uses this characteristic. This means that the laser light sensor unit 126 can accurately identify anomalous emission such as pre-lasing (S-polarized light) by disposing the polarizing plate 110 in front of the power meter, as shown in FIG. 7.

By including this laser light sensor unit 126, the object information acquiring apparatus can reconstruct an image with omitting predetermined data. Predetermined data here refers to data acquired by a laser light 115 that includes anomalous emission such as pre-lasing. This laser light sensor 107d can be an inexpensive undivided single element sensor 109d, hence the object information acquiring apparatus having this sensor 109d can also be created at low cost.

Further, unlike Example 1, the determination unit 123, to determine whether anomalous emission such as pre-lasing is generated or not, is not required. That is, it is unnecessary to determine whether anomalous emission such as pre-lasing is generated or not, because the polarizing plate 110 that transmits only anomalous emission such as pre-lasing is disposed. In other words, anomalous emission can be detected only by determining on whether the sensor 109d detected the light or not. This detection result (detection content) is outputted to the acoustic wave signal processing unit 106 and the laser light source 102. The object information is acquired based on this output. Therefore the object information acquiring apparatus can be manufactured easily and at low cost.

### Example 5

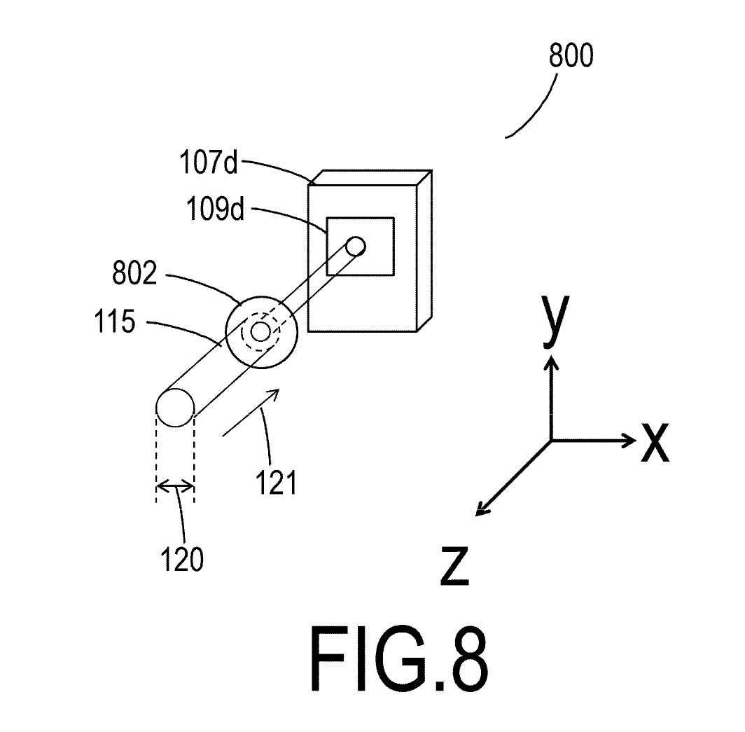

FIG. 8 is a diagram depicting a laser light sensor unit according to Example 5 of the present invention. A composing element the same as Example 1 is denoted with a same number, for which description is omitted unless necessary. The laser light sensor unit 800 according to Example 5 has the same configuration as Example 4, except that an aperture 802, which is a semi-light shielding member, is disposed instead of the polarizing plate 110 in Example 4. The aperture 802 is disposed in the previous stage of a photo acceptance unit 109d. In other words, the laser light 115 reaches the photo acceptance unit 109b via the aperture 802. The photo acceptance unit 109d is a single element sensor that is the same as the one used in Example 4. The aperture 802 has a φ2 mm hole. The aperture 802 is disposed such that a φ2 mm range, which is smaller than the range where the laser light intensity is distributed on a plane perpendicular to the traveling direction of the laser light 115, matches with the above-mentioned hole. In other words, the aperture 802 is disposed on a straight line that passes through the center of the emission energy distribution profile of the laser light 115. The φ2 mm range corresponds to about 20 divided elements in Example 1. This means that the energy when a giant pulse is generated is 3.0 mJ, and the energy when anomalous emission such as pre-lasing is generated is 6.0 mJ. This result is equivalent to the result of FIG. 4 of Example 1. By including this laser light sensor unit 800, an object information acquiring apparatus which has an accuracy equivalent to the laser light sensor 107a of Example 1 can be provided, with good data acquisition performance and having low cost because a single element sensor is used

### Example 6

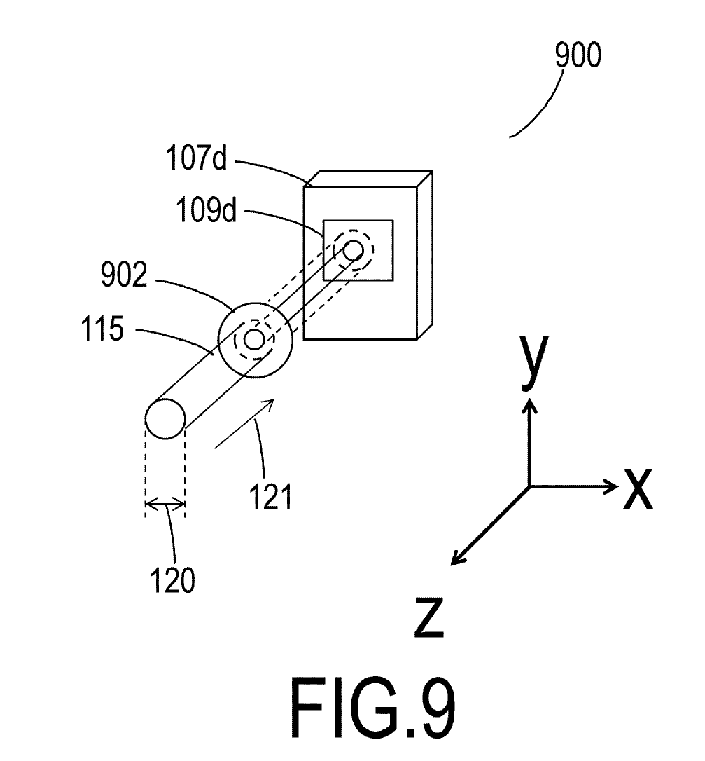

FIG. 9 is a diagram depicting a laser light sensor unit according to Example 6 of the present invention. A composing element that same as Example 1 is denoted with a same number, for which description is omitted unless necessary. Example 6 is the same as Example 5, except that a metal iris 902, of which size of the hole can be changed, is disposed instead of the aperture 802 of Example 5. The iris 902, which is a semi-light shielding member, used for Example 6 is made of metal, and the size of the hole thereof can be changed. This iris is disposed on a straight line that passes through the center of the emission energy distribution profile of the laser light 115. Unlike Example 5, the iris is adjusted to φ2 mm only to detect anomalous emission such as pre-lasing. Thereby anomalous emission such as pre-lasing can be detected with a detectivity equivalent to Example 1. Further, if the size of the hole is changed by changing the aperture of the iris, the detectivity can be appropriately adjusted for other kinds of anomalous emission. Moreover, all intensity levels of normal giant pulse oscillation can be monitored by adjusting the hole to be wider than the range of the spatial distribution of the intensity of the laser light 115. In this way, one laser light sensor unit 900 can play a plurality of functions. As a result, by including this laser light sensor unit 900, an inexpensive information acquiring apparatus can be created.

<Modifications>

The description on each example is merely an example to describe the present invention, and the present invention can be embodied by appropriately making changes or combinations within a range that does not depart from the true spirit of the invention. The above-mentioned processing and means of the present invention can be freely combined as long as technical inconsistencies are not generated. Various characteristics of the present invention are not limited to the above examples, but can be widely applied. The object information acquiring apparatuses according to Example 1 to Example 6 can be implemented using, for example, an information processor having a CPU, memory or the like, that operate according to programs (software). Instead each composing element of the object information acquiring apparatus may be constituted by hardware, such as circuits, that can input/output and compute information.

<Comparative Technique>

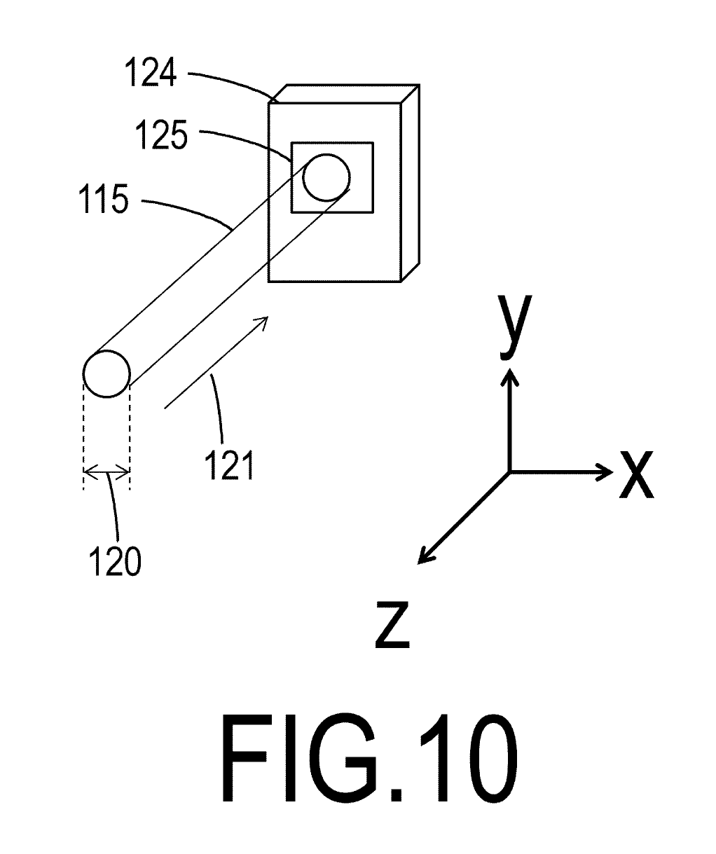

FIG. 10 is a diagram depicting a comparative technique with respect to the present invention. The comparative technique has the same configuration as Example 1, except for the laser light sensor used for Example 1. A laser light sensor 124 used for the comparative technique will now be described. The position of an element unit 125 is the same as the position of the element unit 109a of Example 1 shown in FIG. 3, but unlike the element unit 109a, the element unit 125 is an undivided single element sensor. A case of integrating all the energy in the period of one pulse width using the single element sensor is considered. In this case, the predetermined total light quantity energy is not much different from the total light quantity energy of the single giant pulse emission when anomalous emission such as pre-lasing is not generated. The predetermined total light quantity energy refers to the total light quantity energy that combines both the anomalous emission such as pre-lasing when the anomalous emission is generated and the giant pulse emission.

This means that the laser light sensor 124 of the single element sensor according to this comparative technique cannot determine anomalous emission such as pre-lasing. Therefore in the case of an object information acquiring apparatus using the sensor of this comparative technique, accurate data on the object information cannot be acquired. On the other hand, according to each example of the present invention, an object information acquiring apparatus which can detect anomalous emission such as pre-lasing, and acquire an accurate image where the influence of anomalous emission such as pre-lasing is minimized, can be provided, as described above.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-127527, filed on Jun. 20, 2014, which is hereby incorporated by reference herein in its entirety.

What is claimed is:

1. An object information acquiring apparatus, comprising:
a laser light source configured to irradiate laser light onto an object;
a detector configured to detect a part of the laser light from the laser light source;
a receiver configured to receive an acoustic wave that propagate from the object, based on the irradiation of the laser light; and
an acquisition unit configured to acquire information relating to the object, based on a reception result of the receiver and a detection result of the detector, wherein
the detector detects anomalous emission from the laser light source.

2. The object information acquiring apparatus according to claim 1, wherein
the detector detects pre-lasing.

3. The object information acquiring apparatus according to claim 1, wherein
the acquisition unit acquires information relating to the object by forming an image based on the reception result and the detection result.

4. The object information acquiring apparatus according to claim 3, wherein
for forming the image, the acquisition unit does not use the reception result based on the irradiation acquired when anomalous emission is detected.

5. The object information acquiring apparatus according to claim 1, wherein
the detector detects a part of a spatial distribution, and the part includes a peak of the spatial distribution.

6. The object information acquiring apparatus according to claim 1, wherein
the detector is an area sensor.

7. The object information acquiring apparatus according to claim 1, wherein
the detector is a line sensor.

8. The object information acquiring apparatus according to claim 1, wherein
the detector includes an aperture.

9. The object information acquiring apparatus according to claim 1, wherein
the detector includes an iris.

10. The object information acquiring apparatus according to claim 1, wherein
the laser light source includes a laser medium, two reflectors, a Q switch disposed between the two reflectors, and an excitation unit configured to optically excite the laser medium.

11. The optical information acquiring apparatus according to claim 1, wherein
the anomalous emission has a polarization characteristic which is different from that of a giant pulse irradiated from the laser light source.

12. The optical information acquiring apparatus according to claim 11, further comprising a polarization unit configured to be disposed in a previous stage of the detector, and transmits light having a polarization characteristic which is different from that of the giant pulse.

13. The object information acquiring apparatus according to claim 11, wherein
the polarization unit is a polarizing plate.

14. The object information acquiring apparatus according to claim 11, wherein
the detector is a single light intensity sensor.

15. An object information acquiring apparatus, comprising:
a laser light source configured to irradiate laser light onto an object;
a detector configured to detect a part of the laser light from the laser light source;
a determination unit configured to determine whether anomalous emission is included in the laser light, based on a detection result of the detector;
a receiver configured to receive an acoustic wave that propagate from the object, based on the irradiation of the laser light; and
an acquisition unit configured to acquire information relating to the object, based on a reception result of the receiver and a determination result of the determination unit.

16. An object information acquiring apparatus, comprising:
a laser light source configured to irradiate laser light onto an object;
a detector configured to detect a part of the laser light from the laser light source;
a receiver configured to receive an acoustic wave that propagate from the object, based on the irradiation of the laser light; and
an acquisition unit configured to acquire information relating to the object, based on the reception result of a receiver and a detection result of the detector, wherein
the acquisition unit acquires information on the object using the reception result of the receiver, excluding a reception result, which is acquired when the detection result of the detector indicates anomalous emission.
